SEQUENCE LISTING (12 sequences)

SEQ ID NO: 1 - protein, 2332 aa, Homo sapiens
  human Factor VIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - DNA, 41 bp, Artificial Sequence
  Description of Artificial SequencePCR amplification primer F8Xc2-5
atctctctcgagaaaagagtggatttaaatagttgcagcat

SEQ ID NO: 3 - DNA, 36 bp, Artificial Sequence
  Description of Artificial SequencePCR amplification primer F8NC32
agacagcggccgctagtagaggtcctgtgcctcgca

SEQ ID NO: 4 - DNA, 41 bp, Artificial Sequence
  Description of Artificial Sequence complementary oligonucleotide C2CYS 5′
tcgagaaaagaatgggctgtgatttgaattcttgcagcatg

SEQ ID NO: 5 - DNA, 33 bp, Artificial Sequence
  Description of Artificial Sequence complementary oligonucleotide C2CYS3′
ctgcaagaattcaaatcacagcccattcttttc

SEQ ID NO: 6 - DNA, 9029 bp, Homo sapiens
  human Factor VIII cDNA
  mat_peptide (898)..(7224)
gcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccatttggtcttaattctgaccaataaaaaaataagtcaggaggatgcaattgttgaaagctttgaaataaaataacaatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgaccccttcactatgactacacagaatctcctgatagtaaagggggctggaggcaaggataagttatagagcagttggaggaagcatccaaagattgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacatac

SEQ ID NO: 7 - protein, 160 aa, Artificial Sequence
  Description of Artificial Sequencehuman Factor VIII C2 domain
  VARIANT (20): Xaa = Ala (wild-type) or Pro (hemophilia A mutation)
  VARIANT (35): Xaa = Lys (wild-type) or Gln, Leu or Gly (hemophilia A mutations)
  VARIANT (54): Xaa = Pro (wild-type) or Cys (hemophilia A mutation)
  VARIANT (66): Xaa = Met (wild-type) or Val (hemophilia A mutation)
  VARIANT (73): Xaa = Thr (wild-type) or Ala (hemophilia A mutation)
  VARIANT (74): Xaa = Gln (wild-type) or Arg (hemophilia A mutation)
  VARIANT (90): Xaa = Ile (wild-type) or Thr (hemophilia A mutation)
  VARIANT (111): Xaa = Phe (wild-type) or Val (hemophilia A mutation)
  VARIANT (128): Xaa = Pro (wild-type) or Ser or Leu (hemophilia A mutation)
  VARIANT (132): Xaa = Arg (wild-type) or Gly or Cys (hemophilia A mutation)
  VARIANT (135): Xaa = Arg (wid-type) or Gln or Leu (hemophilia A mutation)
  VARIANT (148): Xaa = Arg (wid-type) or Thr (hemophilia A mutation)
  VARIANT (153): Xaa = Gly (wild-type) or Ser (hemophilia A mutation)
SCSMPLGMESKAISDAQITXSSYFTNMFATWSPSXARLHLQGRSNAWRPQVNNXKEWLQVDFQKTXKVTGVTXXGVKSLLTSMYVKEFLXSSSQDGHQWTLFFQNGKVKVXQGNQDSFTPVVNSLDPXLLTXYLXIHPQSWVHQIALXMEVLXCEAQDLY

SEQ ID NO: 8 - protein, 160 aa, Artificial Sequence
  Description of Artificial Sequencemurine Factor VIII C2 domain
SCSIPLGMESKVISDTQITASSYFTNMFATWSPSQARLHLQGRTNAWRPQVNDPKQWLQVDLQKTMKVTGIITQGVKSLFTSMFVKEFLISSSQDGHHWTQILYNGKVKVFQGNQDSSTPMMNSLDPPLLTRYLRIHPQIWEHQIALRLEILGCEAQQQY

SEQ ID NO: 9 - protein, 160 aa, Artificial Sequence
  Description of Artificial Sequencecanine Factor VIII C2 domain
SCSMPLGMESKAISDAQITASSYLSSMLATWSPSQARLHLQGRTNAWRPQANNPKEWLQVDFRKTMKVTGITTQGVKSLLISMYVKEFLISSSQDGHNWTLFLQNGKVKVFQGNRDSSTPVRNRLEPPLVARYVRLHPQSWAHHIALRLEVLGCDTQQPA

SEQ ID NO: 10 - protein, 160 aa, Artificial Sequence
  Description of Artificial Sequenceporcine Factor VIII C2 domain
SCSMPLGMQNKAISDSQITASSHLSNIFATWSPSQARLHLQGRTNAWRPRVSSAEEWLQVDLQKTVKVTGITTQGVKSLLSSMYVKEFLVSSSQDGRRWTLFLQDGHTKVFQGNQDSSTPVVNALDPPLFTRYLRIHPTSWAQHIALRLEVLGCEAQDLY

SEQ ID NO: 11 - protein, 159 aa, Artificial Sequence
  Description of Artificial Sequenceporcine Factor V C2 domain
GCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDI

SEQ ID NO: 12 - protein, 150 aa, Artificial Sequence
  Description of Artificial Sequencehuman Factor VIII C1 domain
KCQTPLGMASGHIRDFQITASGQYGQWAPKLARLHYSGSINAWSTKEWIKVDLLAPMIIHGIKTQGARQKFSSLYISQFIIMYSLDGHHWQTYRGNSTGTLMVFQGNVDSSGIKHNIFNPPIIARYIRLHPTHYSIRSTLRMELMGCDLN

(19) United States
(12) Patent Application Publication
(10) Pub. No.: US 2006/0293505 A1
(43) Pub. Date: Dec. 28, 2006

(54) Crystal of a truncated protein construct containing a coagulation factor VIII C2 domain in the presence or absence of a bound ligand and methods of use thereof

(76) Inventors: Barry L. Stoddard, Bellevue, WA (US); Kathleen Pratt, Seattle, WA (US); Kazuo Fujikawa, Seattle, WA (US); Earl W. Davie, Bellevue, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/336,487

(22) Filed: Jan. 19, 2006

Related U.S. Application Data
(63) Continuation of application No. 10/049,399, filed on Nov. 4, 2002, now abandoned.
(60) Provisional application No. 60/148,907, filed on Aug. 13, 1999.

(51) Int. Cl. C07K 14/745; G06F 19/00

(52) U.S. Cl. 530/383; 702/19

(57) ABSTRACT

A detailed three-dimensional structure for the C-terminal C2 domain of blood coagulation factor VIII is disclosed. The novel truncated factor VIII constructs which were designed so as to omit a significant portion of the flexible full length protein are also part of the present invention. In addition, the crystals of the protein, both in the presence and absence of bound ligands are also included. Furthermore, methods of identifying antagonists of the human factor VIII protein which can be used to inhibit coagulation or to stabilize and activate factor VIII mutants are also disclosed. Furthermore, methods of identifying variations of the C2 domain sequence and structure that can be incorporated into intact factor VIII for the purpose Of administration to hemophiliac patients who are immunoreactive against wild type factor VIII are disclosed.

### STATEMENT AS TO RIGHTS TO INVENTIONS MADE UNDER FEDERALLY SPONSORED RESEARCH AND DEVELOPMENT

The research leading to the present invention was supported, at least in part, by grants from the National Institutes of Health, Grant Nos. GM49857, HL62470 and HL 16919. The Government may have certain rights in the invention.

### FIELD OF THE INVENTION

The present invention relates to a form of the factor VIII coagulation protein that can be crystallized in the presence or absence of a ligand to form a crystal with sufficient quality to allow detailed crystallographic data to be obtained. The crystals and the three-dimensional structural information are also included in the invention. In addition, the present invention includes procedures for related structure-based drug design and protein engineering using the crystallographic data.

### BACKGROUND OF THE INVENTION

Factor VIII is a plasma protein consisting of 2332 amino acid residues (SEQ ID NO: 1) and is a critical cofactor in hemostasis (FIG. 1). Factor VIII increases the Vₘₐₓ of factor X activation by factor IXa by 200,000-fold in the presence of calcium and negatively-charged phospholipid (see van Diejienk, et al., J. Biol. Chem. 256: 3433-3442 (1992)). This complex is referred to as the “factor IXa/factor VIIIa” or “tenase” complex (see Mann, K. G., et al., Ann. Rev. Biochem. 57: 915-956 (1988) and Kane, Blood 71: 539-555 (1988)). Factor Xa, which is part of a “prothrombinase” complex that is remarkably analogous to the “tenase” complex, then proceeds to convert prothrombin to thrombin. The “tenase” and “prothrombinase” complexes both form at the surface of phospholipid vesicles containing negatively-charged phosphatidylserine in vitro. These vesicles are a model for the in vivo processes that occur at the surfaces of thrombin-activated platelets and damaged endothelium, which transiently expose phosphatidylserine. Deficiencies in factor VIII result in hemophilia A, the most widely-occurring form of hemophilia (Sadler, et al., The Molecular Basis of Blood Diseases: 575 (1987)).

Factor VIII circulates in plasma in a tight (K_{d}=0.52 nM) complex with von Willebrands factor (vWF) (Saenko, et al., J. Biol. Chem. 270: 13826-13833 (1995)). Von Willebrands factor stabilizes and regulates the activity of factor VIII, mediates the attachment of platelets to the subendothelium following vascular injury, and also plays a role in platelet aggregation. Prior to activation by thrombin, factor VIII shows no detectable cofactor activity in the conversion of factor X to the active factor Xa. Physiologically, the major route for the activation of factor VIII is through thrombin-catalyzed cleavage of the precursor factor VIII chain, creating a “heavy” chain and a 73 kD “light chain” (Kaufman, Annu. Rev. Med., 43: 325-339 (1992)). Subsequent cleavages of the heavy chain result in an active heterotrimer stabilized by metal ions. After thrombin cleaves the light chain between residue 1689-1690, the complex with vWF dissociates, and factor VIIIa binds specifically to phosphatidylserine-containing membranes via a binding site at the C-terminus of the light chain (Arai, et al., J. Clin. Invest. 83: 1978-1984 (1989)and Foster, Blood 75: 1999-2004(1990)). Additional thrombin cleavages occur at residues 372-373 and 740-741 (Saenko, et al., J. Biol. Chem. 270: 13826-13833 (1995)). Factor Xa also cleaves at these sites, as well as at 336-337 and 1721-1722, whereas factor IXa cleaves factor VIII at 336-337 and at 1719-1720 (Kane, et al., Blood 71: 539-555 (1988)). Reconstitution of the factor Xa-cleaved light chain resulted in a tenase complex having an association rate constant that was 3× lower than that of thrombin-cleaved or intact light chain, indicating that this cleavage may be significant in the inactivation of the procoagulant complex (Donath, et al., Eur. J. Biochem. 240: 365 (1996)). Sulfated tyrosine residues have been located in recombinant factor VIII adjacent to thrombin cleavage sites, but the functional significance of this modification is not yet clear (Pittman, et al., Thromb. Haemost., 58: 344 (1987)). Factor VIIIa is inactivated by activated protein C in a reaction requiring calcium, the cofactor protein S, and an anionic phospholipid surface (Kane, et al., Blood 71: 539-555 (1988); Esmon, Science 235: 1348-1352 (1987); and Clouse, et al., N. Engl. J. Med 314: 1298-1304 (1986)). The peptide 2009-2018, corresponding to the C-terminal region in the A3 domain has been shown to inhibit the anticoagulant activity of activated protein C (Walker, et al., J Biol. Chem. 265: 1484-1489 (1990)). Factor IXa interacts with factor VIIIa in the regions 558-565 and 698-710 in the A2 domain, and interaction with the light chain is also implied by the inhibition of the binding of IXa by a monoclonal antibody specific for the 1778-1840 region of factor VIII domain A3 (Lenting, et al., J. Biol. Chem. 269: 7150-7155 (1994)). Peptide competition studies have shown that the segment in A3 from 1811-1818 comprises the minimal region required for binding to factor IXa (Lenting, et al., J. Biol. Chem. 271: 1935-1940 (1996)).

The prothrombinase complex has been characterized more extensively than has the tenase complex (Krishnaswamy, et al., Methods Enzymol, 272: 260-280 (1983)), largely because its components occur in higher concentrations in plasma and because factor V is less labile than factor VIII, making purification of the substituents more tractable. In the assembly of the prothrombinase complex, factor Xa and factor Va bind separately to negatively-charged phospholipid vesicles, then diffuse in the vesicle to form an active complex. In the case of factors Xa and Va, the association appears to provoke a conformational change in factor Xa, positioning its active site above the membrane surface at the proper distance and orientation for optimal activity as a part of the prothrombinase complex (Mann, et al., Blood 76: 1-16 (1990)). The tenase complex is thought to carry out its catalytic function in a similar manner, although there are some interesting differences between the two systems. For instance, factor Va will bind to uncharged phospholipid vesicles, whereas factor VIIIa requires negatively-charged phospholipids for membrane attachment.

Factors VIII and V have a similar domain structure; the structure of factor VIII and its thrombin cleavage products are illustrated in FIG. 1b. Unactivated factor VIII is a single peptide chain containing three repeats of a ˜330-residue “A” domain and two repeats of a ˜150-residue “C” domain. The sequence identity between the A domains is approximately 30%, and between the C domains it ranges from 35% to 50% (Kaufman, Annu. Rev. Med. 43: 325-339 (1992) and Jenny, et al., Proc. Natl. Acad. Sci. 84: 4846-4850 (1987)). The A domains also show a ˜30% sequence identity with the copper-binding protein ceruloplasmin, and the C domains have a sequence identity of about 20% with the slime mold lectin discoidin (Poole, et al., J. Mol. Biol. 153: 273-289 (1981)). The large B domains of factors V and VIII contain many Asn-linked glycosylation sites, and show no significant homology with each other. The B domain is removed in the activation of both cofactors, resulting in smaller, multichain proteins having full activity. The purpose of the B domains remains largely elusive, but it is clear that they are fully expendable for the cofactor activity of these proteins (Kane, et al., Blood 71: 539-555 (1988)). Fully processed and activated factor VIIIa is a heterotrimer containing the cleaved peptides from the heavy chain (A1+A2) and a single light chain (A3−C1−C2). The complex of the heavy and light chains contains a single copper atom that was identified using atomic absorption spectroscopy (Bihoreau, et al., C.R. Acad. Sci. 316: 536-539 (1993)). The noncovalent association of the three chains appears to be primarily electrostatic. The isolated subunits do not display factor VIIIa activity, but the separate chains can be combined and reconstituted by dialysis against buffers containing Mn2+, Ca²⁺ or Co²⁺ to form a fully functional factor VIIIa (Nordfang, et al., J. Biol. Chem. 263: 1115-1118 (1988)). Recombinant factor VIII protein has been expressed in hamster kidney cells, and the recombinant protein is structurally and functionally very similar to plasma-purified factor VIII (Eaton, et al., J. Biol. Chem. 262: 3285-3290 (1987))

Experiments utilizing both proteolytic and recombinant fragments of the protein constituents of these complexes indicate that the individual domains of these proteins retain many of their physiologically relevant properties. For example, studies utilizing short peptides derived from the C-terminus of the C2 domain of factor VIII have shown that these peptides compete with factor VIIIa for binding sites on phosphatidylserine-containing phospholipid surfaces (Saenko, et al, J. Biol. Chem. 270: 13826-13833 (1995) and Arai, et al., J. Clin. Invest. 83: 1978-1984 (1989)). Recombinant C2 domain from factor VIII has been expressed in a baculovirus system, and has been shown to compete with factor VIII in binding to a proteolytic fragment of vWF consisting of vWF residues 1-272 (Saenko, et al., J. Biol. Chem. 270: 13826-13833 (1995)). The integrity of the binding site for C2 in the vWF fragment was demonstrated by identical inhibitory effects of C2-derived peptides and of a monoclonal antibody against an epitope in the C2 domain upon complex formation with factor VIII (Saenko, et al., J. Biol. Chem. 270: 13826-13833 (1995)). This same fragment of vWF blocked the binding of factor VIII to immobilized phosphatidylserine (PS), indicating the close juxtaposition of the vWF- and PS-binding sites in the C2 domain of factor VIII. In addition, a monoclonal antibody against an epitope in a different region of the C2 domain showed a similar affinity for factor VIIIa and for the recombinant C2 domain, indicating that the recombinant C2 domain was folded correctly (Saenko, et al., J. Biol. Chem. 270: 13826-13833 (1995)).

The factor VIII mutation database (Wacey, et al., Nucleic Acids 24: 100-102 (1996)) lists 16 mutations in the C2 region that are associated with mild to severe hemophilia A. Recently, an additional eight mutations were added to this list.

The importance of this region for the binding of factor VIII to phospholipids and to vWF has been demonstrated unequivocally (Kane, et al., Blood 71: 539-555 (1988) and Saenko, et al., J. Biol. Chem. 270: 13826-13833 (1995) and Kaufman, et al., Annu. Rev. Med 43: 325-339 (1992)), but the lack of structural information leaves the basis for the effect of these defects unclear. An NMR study of a peptide corresponding to the C2 domain residues 2303-2324 (Gilbert, et al., Biochemistry 34: 3022-3031 (1995)) has indicated that this peptide is disordered in solution, but that it acquires a distinct conformation at pH 6.0 in the presence of SDS micelles, which presumably mimic the interaction of negatively-charged phospholipids with this region. It is also reported that the peptide has an extended conformation from residues 2306-2310, followed by an amphiphilic helix encompassing residues 2310-2322. The peptide competed with fluorescein-labeled factor VIII for binding sites on synthetic PS-containing membranes and on stimulated platelets, with a Kᵢ of 3 μm. Further structural work will characterize the involvement of other regions of factor VIII in binding to phospholipids and to vWF, and aid in understanding the effect of the mutations upon these binding interactions or upon the structural stabilization of the factor VIII molecule. In particular, the three-dimensional structure of the C2 domain would shed light upon the effect of the mutations in the C2 domain that are associated with mild to severe hemophilia A (Wacey, et al., Nucleic Acids Res. 24: 100-102 (1996) and Tuddenham, et al., Nucleic Acids Res. 22: 4851-4868 (1994)).

### SUMMARY OF THE INVENTION

In one aspect, the present invention provides crystals of protein-ligand complexes wherein the protein comprises the N-terminal truncated portion of factor VIII, or a derivative or analog thereof, and the ligand is a negatively charged phospholipid, phosphate or sulfate. Preferably, the protein comprises the C2 domain of human coagulation factor VIII (or a derivative or analog therof), and the ligand is glycerophosphorylserine, which corresponds to the phospholipid head group. Derived from these crystals and related crystals is detailed three-dimensional structural information for the carboxy-terminal C2 domain of human coagulation factor VIII, in the presence and absence of a bound ligand, typically glycerophosphorylserine, phosphate or sulfate.

In another aspect, the present invention provides modified forms of the C2 domain, that are amenable to crystallization and to heavy-metal derivatization, as well as nucleic acids, expression vectors, and cells useful in producing such proteins.

In yet another aspect, the present invention provides methods of identifying antagonists of the C2 domain of human coagulation factor VIII which can be used to regulate or diminish coagulation in mammals, especially humans.

In still another aspect, the present invention provides methods of identifying and analyzing mutant variants of the C2 domain of human coagulation factor VIII that can be incorporated into full length factor VIII, so that hemophiliac patients display reduced or altered immune responses to treatments with factor VIII.

### BRIEF DESCRIPTION OF THE DRAWINGS

FIG. 1A. Molecular associations exhibited by Factor VIII in coagulation. Factor VIIIa increases the Vₘₐₓ of factor X activation by factor IXa by 200,000-fold in the presence of calcium and negatively-charged phospholipid. This complex is referred to as the “factor IXa/factor VIIIa” or “tenase” complex. Factor Xa, which is part of a “prothrombinase” complex that is remarkably analogous to the “tenase” complex, then proceeds to convert prothrombin to thrombin, FIG. 1B: Domain Structure and thrombin cleavage pattern of factor VIII. The factor VIII precursor is activated by thrombin, which cleaves the precursor in several locations and removes the B domain to form factor VIIIa. The binding sites for vWF, phospholipid, and factor IX are shown, as are the primary sites of proteolytic processing.

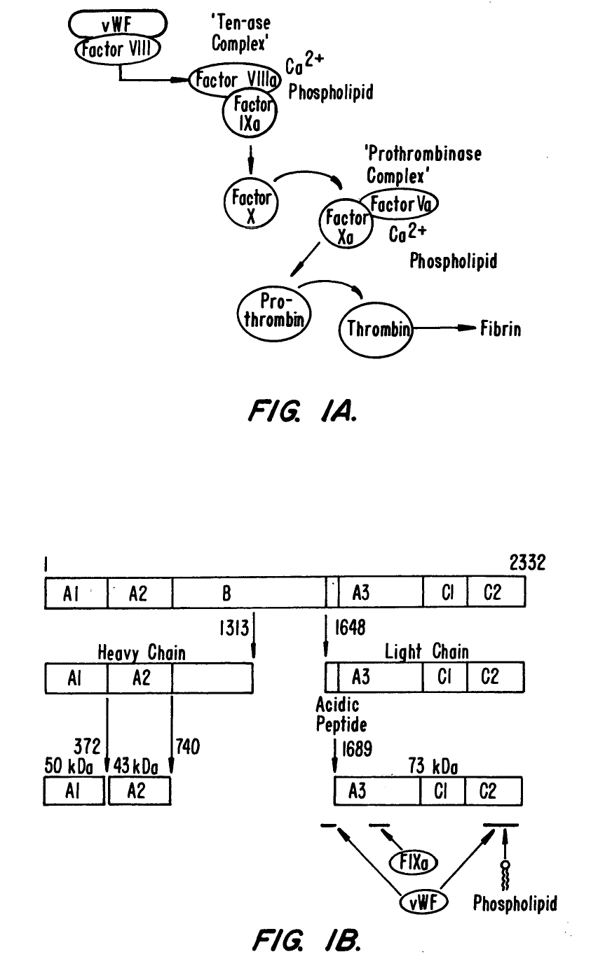

FIG. 2. Primary structure alignments of homologous C domains from factor V and factor VIII. Sites and identities of published hemophilia point mutations are indicated above aligned sequences (SEQ ID NO: 7 through SEQ ID NO: 12); the secondary structure of the human factor VIII C2 domain as determined from the crystal structure is shown below the aligned sequences. Mammalian factor VIII C2 domains are 80 to 90 percent identical, while the human factor V C2 and factor VIII C1 domains both exhibit approximately 40 percent identity to the human factor VIII C2 domain. Positions that are conserved among all six aligned sequences are shown in bold-face. The serine residue in human factor VIII C2 domain at position 2296 corresponds to the position mutated for the purpose of heavy-metal derivatization.

FIG. 3. Ribbon diagram of the human factor VIII C2 domain. The structure reveals a protein domain consisting of 12 β-strands (52% of the protein sequence). The protein contains an eight-stranded β-sandwich core structure formed by β-strands 2, 5, 6, 7, 9, 10, 11 and 12. β-Turns (one between β-strands 3 and 4, and a second between β-strands 6 and 7) and an additional loop (preceding β-strand 5) extend beyond the core fold. These regions flank a pair of positively charged clefts and are predominantly hydrophobic as shown in FIG. 4.

FIG. 4A. Exposed hydrophobic residues on the factor VIII C2 domain. The orientation is the same as FIG. 3. The protein displays two distinct exposed hydrophobic surfaces. The first, at the upper end of the β-sandwich, includes Phe 2275, Tyr 2332 and Leu 2302. The second surface, formed by two β-turns and a loop as described in FIG. 3, includes Met 2199 and Phe 2200 from the first turn, Leu 2251 and 2252 from the second turn, and Val 2223 from the loop. As shown in FIG. 5, these structures extend approximately 10 Å beyond the protein core and flank a pair of positively charged clefts. This structure therefore appears optimal for associating with negatively charged phospholipid membranes. FIG. 4B: The protein is rotated clockwise by approximately 45° relative to panel a, in order to place the hydrophobic residues (Met 2199, Phe 2200, Leu 2151, Leu 2152, and Val 2223) and underlying basic residues (Arg 2215, Arg 2220, Lys 2249 and Lys 2227) along a horizontal axis (grey line) that represents the predicted position of the polar/nonpolar boundary of the phospholipid bilayer.

FIG. 5. Molecular surface of the factor VIII C2 domain, colored by electrostatic potential. Dark=positive, medium=negative, light=uncharged. The left panel is shown in a similar orientation to FIGS. 3 and 4; the right panel is rotated by 900 about the horizontal axis to look directly into the bottom of the molecule. Uncharged non-polar structures formed by the turns and loops described in FIG. 4 are apparent, consisting of Met 2199 and Phe 2200 from turn 1, Leu 2251 and 2252 from turn 2, and Val 2223 from the nearby loop. Tryptophan 2313 also appears to participate in this hydrophobic surface. A ‘ring’ of solvent accessible, positively charged residues lies directly behind these residues, including Lys 2227, Arg 2215, Arg 2220, and Arg 2320.

FIG. 6. Representative hemophilia point mutations placed in the crystal structure of factor VIII C2 domain. Representative side chains are shown that are known to be mutated in hemophilia A patients. The mutated residues correspond to positions buried in the protein core such as Ile 2262, Ala 2192, and Arg 2304 (that are presumably destabilizing upon mutation), positions at the proposed interface with the C1 domain (Pro 2300), and exposed residues (Val 2223, Gln 2213) that presumably interfere with membrane binding or association with von Willebrands factor.

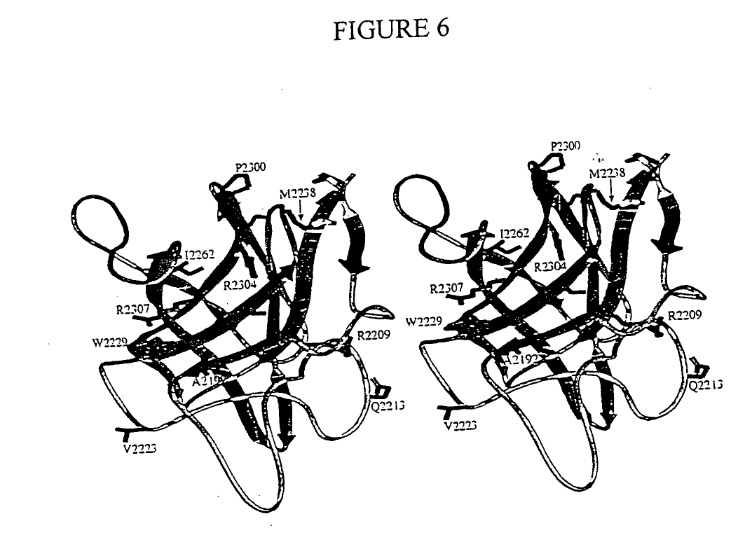

FIG. 7. Target site on C2 domain membrane-binding surface for DOCK screens. The cleft being used for DOCK screens is shown relative to the fold of the protein (left panel), as a shematic with dimensions (middle panel) and as a space-filled diagram (right panel) wherein the proline residue lies at the base of the cleft.

### DETAILED DESCRIPTION OF THE INVENTION

General

The carboxy-terminal C2 domain of human factor VIII binds to exposed phospholipids at sites of vascular damage and initiates coagulation. Mutations in factor VIII, particularly in this domain, are associated with hemophilia A, an often devastating bleeding disorder. Accordingly, crystals of this protein, in the presence of a ligand can provide useful structural information for developing new therapeutic agents, and can also be used in assays to evaluate putative agents. The structure of the human factor VIII C2 domain has now been determined at 1.5 Å resolution. The structure reveals a β-sandwich core structure, from which two β-turns and a loop present a group of solvent-exposed hydrophobic residues that extend beyond an underlying surface of positively charged residues. This region is responsible for association of factor VIII with negatively charged phospholipid membranes. The biological effects of disabling point mutations are correlated with the position of the corresponding side-chain in the protein fold. The structure of the factor VIII C2 domain is similar to the lipid-binding domain.

Description of the Embodiments

I. Crystals of Factor VIII-Ligand Complexes

In one aspect, the present invention provides a crystal of a protein-ligand complex that comprises a complex of N-terminal truncated factor VIII and a ligand. Preferably, the protein is the C2 domain of N-terminal truncated factor VIII and the ligand is glycerophosphorylserine, phosphate or sulfate. In one embodiment, the crystal effectively diffracts X-rays for the determination of the atomic coordinates of the protein-ligand complex to a resolution of greater than 5.0 Angstroms. In a preferred embodiment, the crystal effectively diffracts X-rays for the determination of the atomic coordinates of the protein-ligand complex to a resolution of greater than 3.0 Angstroms. In a more preferred embodiment, the crystal effectively diffracts X-rays for the determination of the atomic coordinates of the protein-ligand complex to a resolution of greater than 2.0 Angstroms. In the most preferred embodiment, the crystal effectively diffracts X-rays for the determination of the atomic coordinates of the protein-ligand complex to a resolution of greater than 1.8 Angstroms.

a. N-Terminal Truncated Factor VIII

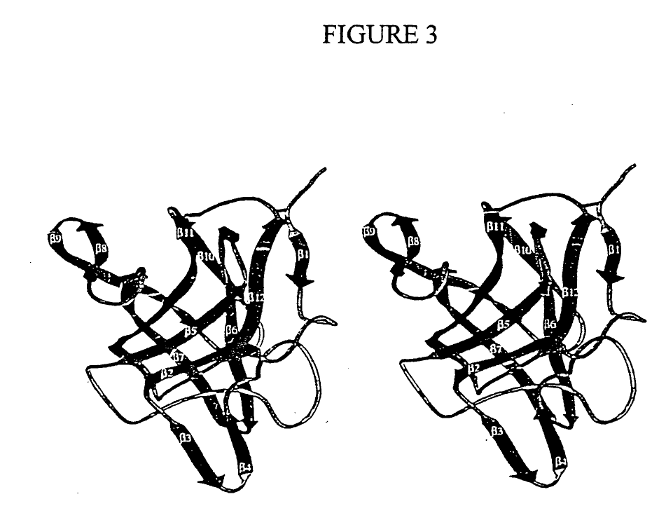

The N-terminal truncated factor VIII used in this aspect of the present invention can be derived from any vertebrate source but is preferably a mammalian Factor VIII, more preferably from a human factor VIII. The N-terminal truncated factor VIII retains the globular core of the C2 domain of factor VIII (see FIG. 3), which is required for (i) the binding of factor VIII to von Willebrands Factor and to negatively charged phospholipids exposed by vascular injury, and (ii) the stimulation of coagulation. The N-terminal truncated factor VIII lacks all or a significant portion (over 2000 amino acids) of the flexible, proteolytically susceptible N-terminal domains of the full-length protein, and can also have a methionine as the initial amino acid prior to the sequence indicated.

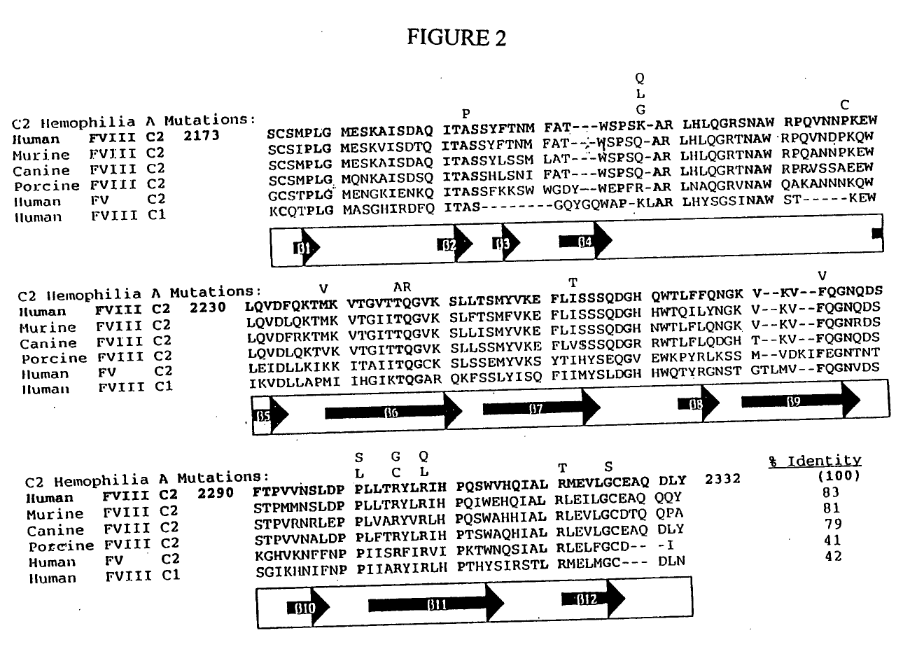

In preferred embodiments the N-terminal truncated factor VIII retains the conserved amino acids depicted in FIG. 2 and consists of approximately 164 amino acids. The N-terminal truncated factor VIII can consist of the wild type sequence shown in FIG. 2, or can possess one or more mutations designed to improve crystallization behavior and/or faclitate derivatization of the protein. In other embodiments, the N-terminal truncated factor VIII can comprise one or more selenomethionines substituted for a naturally occurring methionine of the corresponding factor VIII Of course, general modifications such as additional heavy atom derivatives common in X-ray crystallographic studies may also be performed on the N-terminal truncated factor VIII of the present invention and are included as part of the present invention.

As noted above, in one group of embodiments, the N-terminal truncated factor VIII is derived from full length factor VIII and lacks the first 2000 to 2200 N-terminal amino acids of the corresponding full-length factor VIII As would be evident to one skilled in the art, N-terminal truncated factor VIII may comprise more or less than amino acids 2169 to 2332 of SEQ ID NO:1, but will at least encompass from Cys 2174 to Cys 2326 of SEQ ID NO:1. In one preferred embodiment, the N-terminal truncated factor VIII has an amino acid sequence of amino acids 2169 to 2332 of SEQ ID NO:1, or an amino acid sequence that differs from amino acid 2169 to 2332 of SEQ ID NO:1 by only having conservative substitutions. An example of one such conservative substitution is the replacement of the serine at position 2296 by a cysteine. In another preferred embodiment the N-terminal truncated factor VIII has an amino acid sequence of amino acids 2168 to 2332 of SEQ ID No:1, or an amino acid sequence that differs from amino acid 2168 to 2332 of SEQ ID NO:1 by only having conservative substitutions. Two such conservative substitutions include (i) the incorporation of a cysteine at position 2169, and (ii) the substitution of a cysteine for a serine at position 2296. Consistent with the description above, any of these embodiments can contain one or more selenomethionines in place of a methionine and/or be derivatized with a heavy metal atom.

In still other embodiments, the N-terminal truncated factor VIII used herein can be any of the derivatives and analogs of N-terminal truncated factor VIII described in more detail below.

b. Factor VIII Ligands

The crystals provided in this aspect of the invention will further comprise a ligand that forms a complex with the N-terminal truncated factor VIII. Generally, any ligand that forms a complex with the N-terminal truncated factor VIII can be used to form a crystal of the present invention. Preferably the ligand comprises a negatively charged phospholipid, phosphate or sulfate. More preferably the ligand is glycerophosphorylserine or a derivative thereof.

c. Crystalline Forms of Factor VIII Complexes

A crystal of the present invention may take a variety of forms all of which are included in the present invention. In a preferred embodiment the crystal has a space group of P2₁2₁2₁ and the unit dimensions of about a=46, b=57, and c=66 Angstroms. The N-terminal truncated factor VIII in the crystal has secondary structural elements that include an eight-stranded, antiparallel β-barrel arranged in the order: β-Sheet(1), β-Sheet(2), β-sheet(3), β-sheet(4), β-sheet(5), β-sheet(6), β-sheet(7), β-sheet(8) as depicted in FIG. 3.

Crystals of the N-terminal truncated factor VIII-ligand complex can be grown by a number of techniques including batch crystallization, vapor diffusion (either by sitting drop or hanging drop) and by microdialysis. Preferably, the crystal is grown using sitting-drop vapor diffusion. Seeding of the crystals in some instances is required to obtain X-ray quality crystals. Standard micro and/or macro seeding of crystals may therefore be used.

Once a crystal of the present invention is grown, X-ray diffraction data can be collected. The example below used an RAXIS IV area detector and rotating anode X-ray generator, under standard cryogenic conditions for such X-ray diffraction data collection though alternative methods may also be used. For example, crystals can be characterized by using X-rays produced using a synchotron source. Methods of characterization include, but are not limited to, precision photography, oscillation photography and diffractometer data collection. Heavy-metal derivatives of the crystallized protein can be prepared by soaking or cocrystallizatoin with a number of reactive heavy-metal reagents, including but not limited to mercury and platinum salts. Data can be processed using DENZO and SCALEPACK (Z. Otwinowski and W. Minor). Metal binding sites can be located using SHELXS-90 in Patterson search mode or by visual analysis of Patterson maps. Experimental phases can be estimated via a multiple isomorphous replacement/anomalous scattering strategy using MLPHARE (Z. Otwinowski, Southwestern University of Texas, Dallas). Alternatively, X-PLOR (Brunger, X-PLOR v. 3.1 Manual, Yale University Press, New Haven, Conn. (1992)) or Heavy (T. Terwilliger, Los Alamos National Laboratory) or SHARP may be used. After density modification and non-crystallographic averaging, the protein is built into an electron density map using a program such as O (Jones et al., Acta Cryst., A47: 110-119 (1991)). Model building interspersed with positional and simulated annealing refinement (Brunger, 1993B, supra) can permit the location of the ligand, for example, glycerophosphoserine, and an unambiguous trace and sequence assignment of the N-terminal truncated factor VIII.

II. N-Terminal Truncated Factor VIII and Modified Versions Thereof

In another aspect, the present invention provides N-terminal truncated factor VIII and modified versions thereof, as well as nucleic acids encoding the N-terminal truncated factor VIIIs, expression vectors containing nucleic acids encoding N-terminal truncated factor VIII, and cells transformed or transfected with the expression vectors or nucleic acids described herein. Methods of preparing N-terminal truncated factor VIII and its modified versions are also provided.

The proteins and modified versions thereof are useful in preparing crystals as described above, and are also useful in biochemical screening assays (both cell-based assays and solution assays).

a. N-Terminal Truncated Factor VIII Proteins

In one embodiment the present invention provides an N-terminal truncated factor VIII having an amino acid sequence of amino acids 2169 to 2332 of SEQ ID NO:1 or an amino acid sequence that differs from amino acid 2169 to 2332 of SEQ ID NO:1 by only having conservative substitutions.

The N-terminal truncated factor VIII derivatives of the invention include, but are not limited to, those containing, as a primary amino acid sequence, all or part of the amino acid sequence of an N-terminal truncated factor VIII protein including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. Amino acids containing aromatic ring structures are phenylalanine, tryptophan and tyrosine. The polar neutral amino acids including glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. Particularly preferred substitutions are: Lys for Arg and vice versa such that a positive charge may be maintained; Glu for Asp and vice versa such that a negative charge may be maintained, Ser for Thr such that a free —OH can be maintained, and Gln for Asn such that a free NH₂ can be maintained. Amino acid substitutions may also be introduced to substitute an amino acid with a particularly preferable property. For example, a Cys may be introduced at a potential site for disulfide bridges with another Cys. Pro may be introduced because of its particularly planar structure, which induces β-turns in the protein's structure.

One of skill in the art will understand that certain amino acid residues can be more freely substituted than other amino acids in a conserved region. More specifically, those amino acid residues which map at the surface of an N-terminal truncated factor VIII, as defined by the structural information provided herein, will tolerate even non-conservative changes, and in certain cases, deletions and insertions. Accordingly, the present invention includes all forms of N-terminal truncated factor VIIIs containing conservative and non-conservative changes, provided the protein is functionally equivalent to the wild-type N-terminal truncated factor VIII. As used herein, the term “functionally equivalent,” when applied to the subject proteins, is meant to include all forms of N-terminal truncated factor VIIIs that retain their ability to participate in coagulation cascades and in addition are amenable to being crystallized with a ligand in a crystal that effectively diffracts X-rays for the determination of the atomic coordinates of the protein-ligand complex to a resolution of greater than 5.0 Angstroms.

b. N-Terminal Truncated Factor VIII Nucleic Acids

In another embodiment, a nucleic acid is provided which encodes an N-terminal truncated factor VIII as described above (including those having conserved, and in some instances, non-conserved substitutions). Preferably, the nucleic acid will encode an N-terminal truncated factor VIII having an amino acid sequence of amino acids 2168 to 2332 of SEQ ID NO:1 or an amino acid sequence that differs from amino acid 2168 to 2332 of SEQ ID NO:1 by only having conservative substitutions. The nucleic acid can be derived from natural sources or can be synthesized by solution or solid-phase methods known to those of skill in the art.

(i) Preparation From a Factor VIII Gene

The N-terminal truncated factor VIII proteins, as well as the nucleic acids encoding them, can be prepared from a variety of sources. In a preferred method, a gene encoding factor VIII, including a full length, i.e., naturally occurring form of factor VIII from any organism, can be isolated. Subsequent modification of the coding region of the gene to generate an N-terminal truncated factor VIII can be accomplished according to standard practices. As used herein, the term “gene” refers to an assembly of nucleotides that encode a polypeptide, and includes cDNA and genomic DNA.

A gene encoding factor VIII, whether genomic DNA or cDNA, can be isolated from any vertebrate source, particularly from a human cDNA or genomic library. General methods well known in the art can be used for obtaining factor VIII genes from any source (see, e.g. Sambrook et al., 1989, supra). Accordingly, any vertebrate cell potentially can serve as the nucleic acid source for the molecular cloning of a factor VIII gene. DNA encoding factor VIII may be obtained by standard procedures from cloned DNA (e.g., a DNA “library”), and preferably is obtained from a cDNA library prepared from tissues with high level expression of the factor VIII protein by chemical synthesis, by cDNA cloning, or by the cloning of genomic DNA, or fragments thereof, purified from the desired cell (See, for example, Sambrook et al., 1989, supra; Glover, D. M. (ed.), 1985, DNA cloning: A Practical Approach, MRL Press, Ltd., Oxford U.K. Vol I, II). Clones derived from genomic DNA may contain regulatory and intron DNA regions in addition to coding regions; clones derived from cDNA will not contain intron sequences. Whatever the source, the gene can be molecularly cloned into a suitable vector for propagation.

Propagation of the factor VIII gene can be accomplished using a variety of vector-host systems known in the art. Possible vectors include, but are not limited to, plasmids or modified viruses, as long as the vector system is compatible with the host cell used. Examples of suitable vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coil and Pichia pastoris by linking sequences from an E. coil plasmid with sequences from the yeast plasmid.

In an alternative method, the desired gene may be identified and isolated after insertion into a suitable cloning vector in a “shot gun” approach. Enrichment for the desired gene, for example, by fractionation, can be done before insertion into the cloning vector.

c. Other Factor VIII Nucleic Acid Derivatives

In addition to the nucleic acids described above, the present invention provides nucleic acids which encode functionally equivalent N-terminal truncated factor VIII derivatives.

Factor VIII derivatives can be made by altering encoding nucleic acid sequences by substitutions, additions or deletions that provide for functionally equivalent molecules. Preferably, derivatives are made that are capable of forming crystals of the protein-ligand complex that effectively diffract X-rays for the determination of the atomic coordinates of the protein-ligand complex to a resolution of greater than 5.0 Angstroms.

Due to the degeneracy of nucleotide coding sequences, other DNA sequences which encode substantially the same amino acid sequence as a factor VIII gene may be used in the practice of the present invention. These include but are not limited to allelic genes, homologous genes from other species, and nucleotide sequences comprising all or portions of factor VIII genes which are altered by the substitution of different codons that encode the same amino acid residue within the sequence, thus producing a silent change.

The genes encoding factor VIII derivatives and analogs of the invention can be produced by various methods known in the art. The manipulations which result in their production can occur at the gene or protein level. For example, the cloned factor VIII gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of factor VIII, care should be taken to ensure that the modified gene remains within the same translational stop signals, in the gene region where the desired activity is encoded.

Additionally, the factor VIII-encoding nucleic acid sequence can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Preferably, such mutations enhance the functional activity of the mutated factor VIII gene product. Any technique for mutagenesis known in the art can be used, including but not limited to, in vitro site-directed mutagenesis (Hutchinson, C., et al., J. Biol. Chem. 253:6551 (1978); Zoller and Smith, DNA 3:479-488 (1984); Oliphant et al., Gene 44:177 (1986); Hutchinson et al., Proc. Natl. Acad Sci. U.S.A. 83:710 (1986)), use of TAB^{R} linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

d. Expression Vectors

In addition to the nucleic acids encoding N-terminal truncated factor VIII and its derivatives and analogs, the present invention provides cloning or expression vectors containing genes encoding factor VIII as well as analogs and derivatives of factor VIII including and more preferably the N-terminal truncated factor VIIIs described herein.

The nucleotide sequence coding for factor VIII, an N-terminal truncated factor VIII, derivative or analog thereof, or a functionally active deri vative, including a chimeric protein, thereof, can be inserted into an appropriate expression vector, as described above, which contains the necessary elements, or promoters for the transcription and translation of the inserted protein-coding sequence. Thus, the nucleic acid encoding factor VIII of the invention is operably associated with a promoter in an expression vector of the invention. Both cDNA and genomic sequences can be cloned and expressed under control of such regulatory sequences. An expression vector also preferably includes a replication origin. The necessary transcriptional and translational signals can be provided on a recombinant expression vector, or they may be supplied by the native gene encoding factor VIII and/or its flanking regions.

Typically, the expression vectors comprise a nucleic acid of the present invention operatively associated with an expression control sequence, for example, a promoter. As used herein, the term “expression vector” or “vector” is meant to include a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control. Similarly, the term “cassette” is used in its conventional sense and refers to a segment of DNA that can be inserted into a vector at specific restriction sites. The segment of DNA encodes a polypeptide of interest, and the cassette and restriction sties are designed to ensure insertion of the cassette in the proper reading frame for transcription and translation.

The expression vector will typically be selected to be compatible with a suitable host. Potential host-vector systems include but are not limited to mammalian cell systems infected with virus (e.g., vaccinia virus, adenovirus, etc.); insect cell systems infected with virus (e.g., baculovirus); micro-organisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Transcriptional and translational control sequences are DNA regulatory sequences, such as promoters, enhances, terminators, and the like, that provide for the expression of a coding sequence in a host cell. In eukaryotic cells, polyadenylation signals are DNA regulatory sequences. The expression vectors of the invention comprise an expression control sequence (“promoter” or “enhancer”) in operative association with the nucleic acid or gene. Expression of a factor VIII protein of the invention may be controlled by any promoter/enhancer element known in the art, but these regulatory elements must be functional in the host selected for expression. Choice of suitable regluatory sequences for use in the expression vectors of the invention will be evident to one skilled in the art. Suitable promoters include, but are not limited to, the SV40 early promoter region (Benoist and Chambon, Nature, 290:304-310 (1981)), the promoter contained in the 3′ long terminal repeat of Rous sarcoma virus (Yamamoto et al., Cell, 22:787-797 (1980), the herpes thymidine kinase promoter (Wagner et al., Proc. Natl. Acad Sci. U.S.A., 78:1441-1445 (1981)), the regulatory sequences of the metallothionein gene (Brinster et al., Nature 296:39-42 (1982)); prokaryotic expression vectors such as the P-lactamase promoter (Villa-Kamaroff et al., Proc. Natl. Acad Sci. USA., 75:3727-3731 (1978)), or the tac promoter (DeBoer et al., Proc. Natl. Acad Sci. U.S.A., 80:21-25 (1983)); see also “Useful proteins from recombinant bacteria” in Scientific American, 242:74-94 (1980); promoter elements from yeast or other fungi such as the GAL4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., Cell, 38:639-646 (1984); Ornitz et al., Cold Spring Harbor Symp. Quant. Biol., 50:399-409 (1986); MacDonald, Hepatology, 7:425-515 (1987)); insulin gene control region which is active in pancreatic beta cells (Hanahan, Nature, 315:115-122 (1985)), immunoglobulin gene control region which is active in lymphoid cells (Grosschedl et al., Cell, 38:647-658 (1984); Adames et al., Nature, 318:533-538 (1985); Alexander et al., Mol. Cell. Biol., 7:1436-1444 (1987)), mouse mammary tumor virus control region which is active in testicular, breast, lymphoid and mast cells (Leder et al., Cell, 45:485-495 (1986)), albumin gene control region which is active in liver (Pinkert et al., Genes and Devel., 1:268-276 (1987)), alpha-fetoprotein gene control region which is active in liver (Krumlauf et al., Mol. Cell. Biol. 5:1639-1648 (1985); Hammer et al., Science, 235:53-58 (1987)), alpha 1-antitrypsin gene control region which is active in the liver (Kelsey et al., Genes and Devel., 1:161-171 (1987)), beta-globin gene control region which is active in myeloid cells (Mogram et al., Nature, 315:338-340 (1985); Kollias et al., Cell, 46:89-94 (1986)), myelin basic protein gene control region which is active in oligodendrocyte cells in the brain (Readhead et al., Cell, 48:703-712 (1987)), myosin light chain-2 gene control region which is active in skeletal muscle (Sani, Nature, 314:283-286 (1985)), and gonadotropic releasing hormone gene control region which is active in the hypothalamus (Mason et al., Science, 234:1372-1378 (1986)). In a preferred embodiment of the invention, a Pichia pastoris alcohol oxidase promoter is used to control expression of N-terminal truncated factor VIII proteins of the invention. Within one preferred embodiment the Pichia pastoris AOX1 gene promoter is used.

In a preferred embodiment of the invention, the proteins of the invention are directed into the secretory pathway of the host cell to permit isolation of the expressed protein from the conditioned media Genes encoding the proteins of interest are operably joined to at least one signal sequence. As would be evident to one skilled in the are, the signal sequence may be derived from the factor VIII coding sequence or may include one of many suitable secretory signal including the Saccharomyces cerevisiae alpha-factor secretion signal, the S. cerevisaie BAR1 signal sequence and the like. Within one preferred embodiment, the S. cereviseiae alpha factor secretion signal is used to direct the section of the N-terminal truncated factor VIII proteins.

Any of the methods previously described for the insertion of DNA fragments into a cloning vector may be used to construct expression vectors containing a gene consisting of appropriate transcriptional/translational control signals and the protein coding sequences. These methods may include in vitro recombination DNA and synthetic techniques and in vivo recombination (genetic recombination).

Expression vectors containing a nucleic acid encoding a factor VIII of the invention can be identified by four general approaches: (1) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g. β-galactosidase activity, thymidine kinase activity, resistance to antibiotics, transformation phenotype, occlusion body formation in baculovirus, etc.) caused by the insertion of foreign genes in the vector. In another example, if the nucleic acid encoding factor VIII is inserted within the “selection marker” gene sequence of the vector, recombinants containing the factor VIII insert can be identified by the absence of the factor VIII gene function In the fourth approach, recombinant expression vectors can be identified by assaying for the activity, biochemical, or immunological characteristics of the gene product expressed by the recombinant, provided that the expressed protein assumes a functionally active conformation.

Vectors are introduced into the desired host cells by methods known in the art, e.g., transfection, electroporation, micro-injection, transduction, cell fusion, DEAE dextran, calcium phosphate precipitation, lipofection (lyosome fusion), use of a gene gun, or a DNA vector transporter (see, e.g., Wu, et al., J. Biol. Chem., 267:963-967 (1992); Wu and Wu, J. Biol. Chem., 263:14621-14624 (1988); Hartmut et al., Canadian Patent Application No. 2, 012,311, filed Mar. 15, 1990).

e. Cells Transfected With Factor VIII Expression Vectors

In another embodiment, the present invention provides a cell transfected or transformed with an expression vector of the present invention. Suitable host cells include mammalian, avian, plant, insect and fungal cells. In one embodiment of the invention the cell is a eukaryotic cell. In one such embodiment the eukaryotic cell is a Pichia pastoris cell.

f. Methods of Expressing N-Terminal Truncated Factor VIII

The present invention also includes methods of expressing the N-terminal truncated factor VIII comprising culturing a cell that expresses the N-terminal truncated factor VIII in an appropriate cell culture medium under conditions that provide for expression of the protein by the cell. Any of the cells mentioned above may be employed in this method. In a particular embodiment the cell is a Pichia pastoris cell which has been manipulated to express an N-terminal truncated factor VIII of the present invention. In a preferred embodiment, the method further includes the step of purifying the N-terminal truncated factor VIII.

III. Screening Methods

In yet another aspect, the present invention provides methods of using a crystal or crystal structure of the present invention in a drug screening assay.

In one embodiment, the method comprises selecting a potential ligand by -performing structure-based drug design with a three-dimensional structure determined for the crystal, preferably in conjunction with computer modeling. Such computer modeling is preferably performed with a docking program. The potential ligand is then contacted with the ligand binding domain of factor VIII and the binding of the potential ligand and the ligand binding domain is detected. A potential ligand is selected as a potential drug on the basis of its binding to the ligand binding domain of factor VIII with a similar affinity for the ligand binding domain of factor VIII than a standard ligand, such as glycerophosphorylserine or phosphatidylserine containing vesicles.

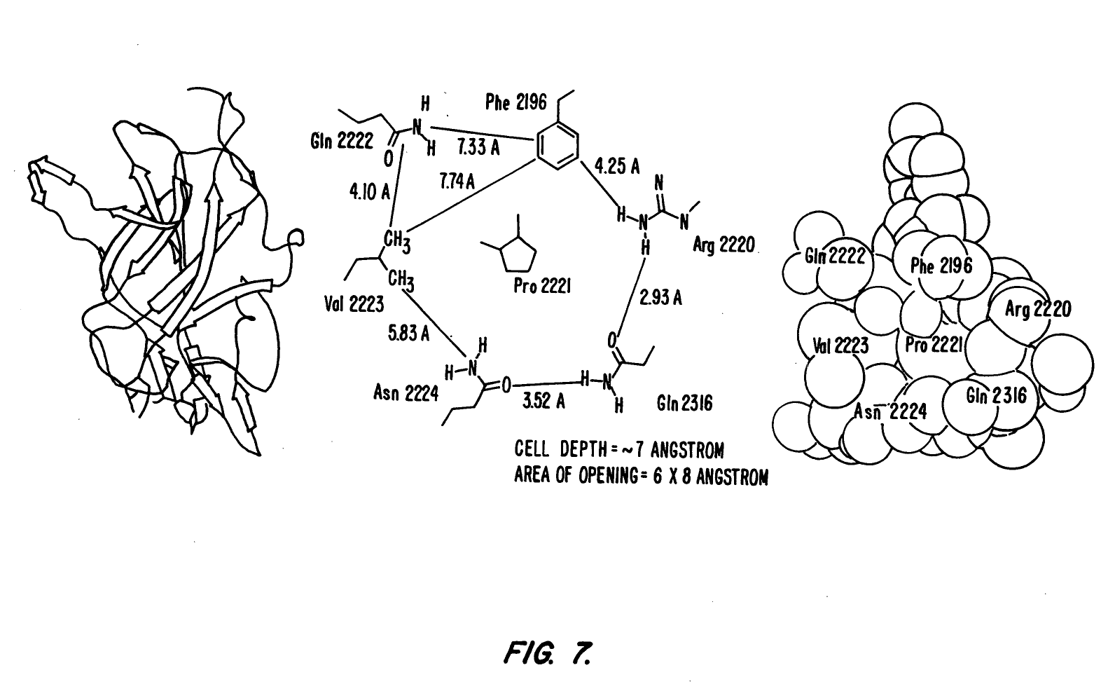

For example, the DOCK program can identify a target site, develop a 3-dimensional model of that site and compare that model to 3-dimensional models of superimposed candidate ligands. The program can then be used to calculate scoring grids that assess and quantitate the potential interaction energy of those candidate ligands to the site. In this manner, a cleft in the approximate center of the membrane-binding surface of FVIII C2, flanked by the hydrophobic β-hairpin turns was identified as an attractive target for ligand screening (see FIG. 7). A molecular surface representation of the cleft was generated using MIDAS. A sphere set (which fills the binding site and represents its “negative” 3-dimensional image) was then generated using the DOCK SPHGEN routine. A ‘dotlim’ value (which defines how finely local invaginations of the molecular surface are sampled) of −1 was used. The maximum sphere radius was 4.0 Å and the minimum was 1.4 Å. The maximum distance between intra-ligand and intra-receptor points was set to 0.25 Å. Precalculated energy grids, used to calculate the energy score for any ligand-receptor atom pair in the docked solutions, were generated by the DOCK routine ‘GRID’ version 4.0. Atomic partial charges were assigned to the receptor site prior to calculating these grids using the MOPAC semi-empirical quantum mechanics package in QUANTA (see Molecular Simulations Inc., San Diego, Calif.). Hydrogen atoms were assigned to the receptor site for assignment of grid partial charges and van der Waals radii. Hydrogen atoms were built with the protein design application in QUANTA. The intermolecular interaction energies were modeled using a combined van der Waals and electrostatic interaction potential. Electrostatic interactions were modeled using a distance dependent dielectric and an initial dielectric constant of 4.

DOCK version 4.0.1 can be used to screen against molecules in the latest release of the Available Chemicals Database (ACD, currently the 1999 release, see Ewing and Kuntz, J. Comp. Chem. 18:1175-1189 (1997)). Three dimensional conformations for each molecule in the ACD are generated using the rule based structure prediction program CONCORD. The ACD currently contains 570,000 unique compounds in 23 individual files. Each file contains an average of 25,000 compounds. Compounds are separated by net charge and the total charge of compounds screened will typically be from 0 to ±4. Additionally, compounds selected for screening will typically have from 10 to 35 heavy atoms (other than hydrogen). Following selection of appropriate candidate compounds, the candidates are screened twice—once using both electrostatic and van der Waals terms, and a second time using only van der Waals interaction terms as a test for shape complementarity. Candidate ligands selected from the ACD are preferably rigid molecules due to computational time constraints. A maximum of 100 orientations are generated for each candidate ligand and the potential energy of each orientation is minimized using, for example, the default SIMPLEX minimizer in DOCK. Suitable candidate ligands that are identified using the computational methods can then be further evaluated using membrane-binding interference assays.

In another embodiment, a supplemental crystal is grown which comprises a protein-ligand complex formed between an N-terminal truncated factor VIII and the potential drug identified above. Preferably the crystal effectively diffracts X-rays for the determination of the atomic coordinates of the protein-ligand complex to a resolution of greater than 5.0 Angstroms, more preferably greater than 3.0 Angstroms, and even more preferably greater than 2.0 Angstroms. The three-dimensional structure of the supplemental crystal is determined by molecular replacement analysis or multi-wavelength anomalous dispersion or multiple isomorphous replacement. A candidate drug is selected or identified by performing structure-based or rational drug design (including binding site optimization studies) with the three-dimensional structure determined for the supplemental crystal, preferably in conjunction with computer modeling. The candidate drug is then contacted with intact or N-terminal truncated factor VIII and a measure of binding to specific phospholipids such as phosphatidylserine is detected. A candidate drug is identified as a drug when it inhibits protein binding to negatively charged phospholipids.

In another embodiment, the present invention provides a method of using a crystal of the present invention in a drug screening assay to identify a candidate drug that inhibits coagulation. In this method, a potential antagonist to factor VIII is identified by performing structure-based drug design with a three-dimensional structure determined for the crystal, preferably in conjunction with computer modeling. The potential antagonist is then added to a coagulation assay in which factor VIII can be the limiting protein factor. A measure of coagulation is determined, and a candidate drug is identified as that compound which inhibits coagulation. The assay can be an in vitro or in vivo assay, but is preferably an in vitro assay. In one such embodiment of this type the assay is performed using human plasma that may or may not be depleted of specific clotting factors, and specific factors and/or drug candidates are added to the assay mixture.

In each of the embodiments above, a supplemental crystal can be grown which comprises a protein-ligand complex formed between an N-terminal truncated factor VIII and the potential (or candidate) drug. Preferably the crystal effectively diffracts X-rays for the determination of the atomic coordinates of the protein-ligand complex to a resolution of greater than 5.0 Angstroms, more preferably greater than 3.0 Angstroms, and even more preferably greater than 2.0 Angstroms. The three-dimensional structure of the supplemental crystal is determined by molecular replacement analysis or multi-wavelength anomalous dispersion or multiple isomorphous replacement. A potentially optimized candidate drug can then be selected by performing structure-based drug design with the three-dimensional structure determined for the supplemental crystal, preferably in conjunction with computer modeling. An optimized candidate drug is identified as a drug when it inhibits binding of factor VIII to specific phospholipids, or when it inhibits coagulation. One of skill in the art will appreciate that in all of the drug screening assays provided herein, a number of iterative cycles of any or all of the steps may be performed to optimize the selection. Additional steps to identify candidate drugs are also contemplated. For example, in one particular embodiment, the potential (or candidate) drug is administered into an animal subject.

In the embodiments above, initial computer modeling can be performed with one or more of the following docking computer modeling programs: DOCK, GRAM, and AUTODOCK, or similar computer programs.

For example, once the three-dimensional structure of a crystal comprising a protein-ligand complex formed between an N-terminal truncated factor VIII and a standard ligand for factor VIII is determined, a potential ligand is examined through the use of computer modeling using a docking program such as GRAM, DOCK, or AUTODOCK (Dunbrack et al., Protein Sci. 6:1661-1681 (1997)), to identify potential ligands and/or antagonists for factor VIII. This procedure can include computer fitting of potential ligands to the ligand binding site to ascertain how well the shape and the chemical structure of the potential ligand will complement the binding site. (Bugg et al., Scientific American, 269:92-98 (1993)); West et al., TIPS, 16:67-74 (1995)). Computer programs can also be employed to estimate the attraction, repulsion, and steric hindrance of the two binding partners (i.e., the ligand-binding site and the potential ligand). Generally the tighter the fit, the lower the steric hindrances, and the greater the attractive forces, the more potent the potential drug since these properties are consistent with a tighter binding constant. Furthermore, the more specificity in the design of a potential drug the more likely that the drug will not interact as well with other proteins. This will minimize potential side-effects due to unwanted interactions with other proteins.

Initially potential ligands and/or agonists can be selected for their structural similarity to phosphatidylserine, a natural phospholipid binding partner to factor VIII. One such example is glycerophosphorylserine which was used in the Example below. The structural analog can then be systematically modified by computer modeling programs until one or more promising potential ligands are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)). A similar analysis could be carried out with the uncomplexed protein structure by targeting putative membrane binding sites such as basic or hydrophobic patches. The final drug candidate may have a similar structure that differs significantly from glycophosphoserine.

Additional computational methods can also be applied to the present invention. For example, the three-dimensional structure of a protein-ligand complex of an N-terminal truncated human factor VIII and a ligand (e.g., the structure disclosed in the example below) can be used to determine the three-dimensional structure of a protein-ligand complex of a second N-terminal truncated factor VIII (e.g., a rat factor VIII ) and a ligand by computer analysis with a computer program that analyzes molecular structure and interactions. Preferably, the computer analysis is performed with one or more of the following computer programs: QUANTA, CHARMM, INSIGHT, SYBYL, MACROMODEL and ICM. More preferably, these computational comparison methods are used in conjunction with the docking programs described above to identify potential or candidate agents.

Once a potential ligand or a potential antagonist is identified it can be either selected from a commercially available library of chemicals or alternatively, the potential ligand or antagonist can be synthesized de novo. The potential ligand can be placed into a standard binding assay with the C2 domain of the factor VIII. Alternatively the N-terminal truncated factor VIIIs or the corresponding full-length proteins may be used in these assays.

For example, the C2 domain of a factor VIII can be attached to a solid support. Methods for placing the ligand binding domain on the solid support are well known in the art and include such approaches as linking biotin to the ligand binding domain and linking avidin to the solid support. The solid support can be washed to remove unreacted species. A solution of a labeled potential ligand can be contacted with the solid support. The solid support is washed again to remove the potential ligand not bound to the support. The amount of labeled potential ligand remaining with the solid support and thereby bound to the ligand binding domain may be determined. Alternatively, or in addition, the dissociation constant between the labeled potential ligand and the ligand binding domain can be determined, Suitable labels include enzymes, fluorophores (e.g., fluorescence isothiocyanate (FITC), phycoe (PE), Texas red (TR), rhodamine, free or chelated lanthanide series salts, especially Eu³⁺, to name a few fluorophores), chromophores, radioisotopes, chelating agents, dyes, colloidal gold, latex particles, nitroxide spin labels, ligands (e.g., biotin), and chemiluminescent agents. When a control marker is employed, the same or different labels may be used for the receptor and control marker.

In the instance where a radioactive label, such as the isotopes ³H, ¹⁴C, ³²P, ³⁶Cl, ⁵¹ Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme detection may be accomplished by any of the presently utilized calorimetric, spectrophotometric, fluorospectrophotometric, amperometric or gasometric techniques known in the art.

Direct labels are also useful in this aspect of the invention. A “direct label” as used herein, is an entity, which in its natural state, is readily visible, either to the naked eye, or with the aid of an optical filter and/or applied stimulation, e.g., U.V. light to promote fluorescence. Among examples of colored labels, which can be used according to the present invention, include metallic particles, for example, gold particles such as those described by Leuvering (U.S. Pat. No. 4,313,734); dye particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, and urease (see, for example, Engvall in Enzyme Immunoassay ELISA and EMIT in Methods in Enzymology, 70:419-439 (1980) and in U.S. Pat. No. 4,857,453).

When suitable potential ligands and/or antagonists are identified, a supplemental crystal is grown which comprises a protein-ligand complex formed between an N-terminal truncated factor VIII and the potential drug. Preferably the crystal effectively diffracts X-rays for the determination of the atomic coordinates of the protein ligand complex to a resolution of greater than 5.0 Angstroms, more preferable greater than 3.0 Angstroms, and even more preferably greater than 2.0 Angstroms. The three-dimensional structure of the supplemental crystal is determined by Molecular Replacement Analysis. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: X-PLOR (see above) and AMORE (J. Navaze, Acta Crystallographics ASO, 157-163 (1994)). Once the position and orientation are known an electron density map can be calculated using the search model to provide X-ray phases. Thereafter, the electron density is inspected for structural differences and the search model is modified to conform to the new structure. Using this approach, it will be possible to use the claimed structure of the mouse factor VIII to solve the three-dimensional structures of any factor VIII having a pre-ascertained amino acid sequence and/or corresponding factor VIII-ligand structures (e.g., containing glycerophosphoserine). Other computer programs that can be used to solve the structures of the factor VIIIs from other organisms include: QUANTA, CHARMM, INSIGHT, SYBYL, MACROMODE, and ICM.

For all of the drug screening assays described herein further refinements to the structure of the drug will generally be necessary and can be made by the successive iterations of any and/or all of the steps provided by the particular drug screening assay.

IV. N-Terminal Truncated Factor VIII Mutants

In yet another aspect, the present invention provides methods of identifying and analyzing mutant variants of the C2 domain of human coagulation factor VIII that can be incorporated into full length factor VIII. Such variants find particular use in treating hemophiliac patients who display reduced or altered immune responses to treatments with factor VIII.

In this method, N-terminal truncated factor VIIIs are used which retain their ability to function as coagulation factors. Typically, the N-terminal truncated factor VIIIs having conservative substitutions in their amino acid sequence are useful, as well as other mutant forms prepared and evaluated as described herein.

The N-terminal truncated factor VIII derivatives and analogs (or other functionally equivalent mutant forms) can be expressed as described above. When expressed in P. pastorisi, the protein is formed as a soluble stable protein product. One such detailed protocol is provided in the Example below. The expressed protein can be purified to homogeneity by standard methods of separative chromatography and then assayed to determine whether it can serve as a functional factor VIII by measuring binding to von Willebrands Factor and/or binding to specific phospholipids.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins,. eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE

In the Example below, the current refinement model consists of factor VIII residues 2169 to 2332 plus glycerophosphorylserine (complex 1), and factor VIII residues 2169 to 2332 in the absence of a bound organic ligand, and 194 water molecules. The electron density for the polypeptide backbone is everywhere continuous at 1.3 σ in a (2|F_{observed}|−|F_{calculated}|) difference Fourier synthesis. PROCHECK (Laskowski et al., J. Appl. Cryst., 26:283-290 (1993)) revealed main-chain and side-chain parameters appropriate for 1.5 Angstrom resolution (overall G-factor=0.15).

Protein Subcloning, Expression and Purification:

Recombinant wild-type and mutant factor VIII C2 domains comprising residues 2169 to 2332 were expressed in Pichia pastoris using the vector pPIC9K (Invitrogen, San Diego, Calif.). The pPIC9K vector contains an expression cassette consisting of the AOX1 promoter, the S. cerevisiae α-factor signal sequence a multiple cloning site and a transcription terminator. In addition to the expression cassette, the vector contains the HIS4 and kanimycin resistance genes for selection purposes. Expression of proteins from cDNAs inserted, in-frame, into this cassette are secreted from the transformed Pichia pastoris cells into the media.

A first wild-type factor VIII C2 domain (the factor VIII cDNA sequence is shown in SEQ ID NO:6) was constructed by PCR amplification of a human factor VIII cDNA as a template (provided by Dr. Ezban, Novo Nordisk, Copenhagen, Denmark) using primers F8Xc2-5 (5′-ATCTCTCTCGAGAAAAGAGTGGATITAAATAGTTGC AGCAT-3′ (SEQ ID NO:2)) and F8NC32 (5′-AGACAGCGGCCGCTAGTAGAGGTCC TGTGCCTCGCA-3′ (SEQ ID NO:3)). The resulting in an amplimer, termed Val-C2, encoding a sequence containing residues Cys 2169 to Tyr 2332 of SEQ ID NO:1 with Cys 2169 replaced with Val. The amplimer was subcloned into pPIC9 vector (Invitrogen, San Diego, Calif.) at the Xho I ahd Not I sites. The DNA fragment comprising the amplimer, designated Val-C2, was isolated from the subcloned pPIC9 vector by Bam HI and Not I digestion and subcloned into the final vector pPIC9K (Invitrogen). The Bam HI-Not I fragment was also subcloned into pUC18 plasmid to form the pUC18Val-C2 vector for the preparation of other constructs. The expression vector containing the Val-C2 amplimer was expressed and purified from Pichia pastoris as generally described herein. However, while the recombinant protein is functional as determined by the methods herein and the protein crystallized, the crystal could not be derivatized.

Based on analysis of the protein sequence, mutant constructs of the factor VIII C2 domain were constructed containing single free cysteine residues to permit heavy metal derivatization and generation of phases. A first mutant was generated to reincorporate a cysteine at position 2169, complementary oligonucleotides, C2CYS 5′ (5′ TCGAGAAAAGAATGGGCTGTGATTTGAATTCTTGCAGCATG-3′ (SEQ ID NO:4)) and C2CYS3′ (5′-CTGCAAGAATTCAAATCACAGCCCATTCTTCTTTTC-3′ (SEQ ID NO:5)) were designed to, when annealed, replace the Xho I-Sph I fragment containing the Val 2169 of the Val-C2 amplimer in plasmid pUC18-C2. Following synthesis and phosphorylation using T4 DNA ligase, the oligonucleotides were annealed and subcloned into the Xho I-Sph I vector fragment of pUC18Val-C2 to create pUC18Cys-C2 vector. The plasmid, pUC18-Cys-C2, was digested with Bam HI and Not I to isolate the fragment encoding Cys-C2. The Barn HI-Not I Cys2-C2 fragment was cloned into expression vector pPIC19K. The expression vector encoding the Cys2-C2 factor VIII C2 domain was expressed and purified from Pichia pastoris as generally described herein. However, while the recombinant protein is functional as determined by the methods herein, protein crystals were not obtained.

A second mutation, termed S2296C, which places a cysteine residue at a position predicted to reside in a surface loop (36 residues from the C-terminus) was generated by designing and synthesizing complementary oligonucleotides that, when annealed result in a fragment encoding a portion of the C2 domain flanking residue 2296 and wherein residue 2296 is a cysteine. The annealed oligonucleotides additionally contain suitable sites at the 5′ and 3′ ends for subcloning into the pUC18Val-C2 construct resulting in a factor VIII mutant C2 domain encoding a protein with Val 2169 and Ser 2296 to Cys 2296 mutations. The mutant S2296C-C2 factor VIII sequence was subcloned into pPIC19K.

The sequences of the coding regions of all expression vectors were confirmed by dideoxy-terminator sequencing (Sanger et al., Proc. Natl. Acad. Sci. USA 74:5463-5467 (1997)).

Expression vectors were linearized by Sac I digestions and transformed into methylotrophic yeast Pichia pastoris strain GS115 (Invitrogen, San Diego, Calif.) by electroporation according to the manufacturer's instructions at 12,500 V/cm, 25 μF, 400 Ω. Integration of the plasmid permits selection of His⁺, G418 resistant transformants. His+ multi-copy transformants were selected on MD plates containing 2.0 mg/ml G418 (see Scorer, et al., Biotechnology (NY) 269:181-184 (1994)). Three different clones were selected for each construct, and the cells were cultured for 2 days in 25 ml of BMGY medium (1% yeast extract, 2% peptone, 100 mM potassium phosphate (pH 6.0), 1.34% YNB, 4×10⁻⁵% biotin, 0.5% glycerol). Cells were then spun down and resuspended in 30 ml of BMMY-3X YP medium (0.1 M potassium phosphate buffer (pH 6.0), 3% yeast extract, 6% peptone, 1.34% yeast nitrogen base, 4×10⁻⁵% biotin, 0.5% methanol; from Invitrogen). The cells were shaken in 250 ml baffled flasks and 300 μl of methanol was added every 24 hours to maintain induction of protein expression.

The culture supernatants were obtained by centrifugation after three days of induction. Ammonium sulfate was added to 45% saturation. Pellets were collected by centrifugation and were dissolved in 50 mM HEPES (pH 7.6), 25 mM NaCl, 1 mM N-ethylmaleimide or 1.0 mM E-64 (trans-Epoxysuccinyl-L-leucylamido-(4-guanidino)butane; Sigma, St. Louis, Mo.), 1 mM DFP (diisopropyl fluorophosphate, Sigma) or PMSF (phenylmethylsulfonyl fluoride, Sigma), 10 μg/ml pepstatin A and 5 mM EDTA. The samples were clarified by centrifugation at 10,000 g at 4° C. for 10 minutes. Samples were then dialyzed against 50 mM HEPES, pH 7.6, 25 mM NaCl and filtered through a 0.45 μM membrane.

The C2 proteins were loaded onto a CM column and eluted with a 0-0.4 M NaCl gradient. The yield of pure wild-type C2 protein was about 5 mg per liter of culture, while yield of mutant proteins (see below) were somewhat lower. The C2 proteins were analyzed by N-terminal amino acid sequencing, mass spectrometry, and by dot blot analysis using a monoclonal antibody specific for the factor VIII C2 domain (The ESH08 antibody (from American Diagnostica, Greenwich, Conn.) recognizes an epitope corresponding to residues 2248-2285 in factor VIII). The functionality of the PS-binding region was initially demonstrated by binding of the protein to microtiter plates coated with phosphatidylserine. The C2 protein did not bind to control plates coated with phosphatidylcholine.

Functional Assays: Binding of Recombinant C2 Domain to Phospatidylserine and Von Willebrands Factor:

Several initial studies clearly indicated that the recombinant C2 domain from factor VIII is properly folded and functional. The protein was expressed as a soluble product and displayed excellent solution properties when concentrated to milligram per milliliter concentrations. The protein was readily crystallized, indicating that it possesses a stable, unique fold. As described above, the protein binds to phosphatidylserine-coated microtiter plates, whereas it does not bind to an analogous surface coated with phosphatidylcholine. Additionally, gel filtration studies (described below) of the protein mixed with different phospholipids show that the protein coelutes with the PS/PC fraction. Together, these studies indicate that this construct retains at least one of the important binding properties associated with its role in the intact protein: specific binding to PS lipid headgroups.

The gel-filtration assays were carried out on a 0.5×19 cm Sephacel-4B column. The following were mixed together:
250 μl PS or PS/PC phospholipid
50 μl Cys2-C2 (4.8 mg/ml, MW 19,000)
50 μL bovine serum albumin (10 mg/ml, MW 68,000)
150 μl HEPES buffer (pH 7)

BSA was added as a control protein, to rule out nonspecific binding to phospholipids. The buffer was 50 mM HEPES, pH 7.4, 0.1 M NaCl, and fractions were collected at 10 drops/tube. Protein in the fractions was determined by Bradford assay, and PS elution was monitored by fluorescent signal at 320 nm, 90° C.

BSA passed through the column separately from the phospholipid fraction, but the C2 protein was found to co-elute with the phospholipid fraction, demonstrating an association between the C2 protein and phospholipid.

Crystallography:

The structure of the protein was solved by multiple isomorphous replacement, using a mutant construct containing a single free cysteine residue for the purpose of mercury derivatization. This mutant (S2296C), places a cysteine residue at a position predicted to reside in a surface loop 36 residues from the C-terminus based on analysis of the protein sequence by the program DSSP. Crystals of the recombinant S2296C-C2 protein were grown from 1.3 M ammonium sulfate, 0.1 M MES (pH 6.0), protein 6 mg/mil, and then frozen after transfer to a cryobuffer of similar composition containing 30% glucose w/v and 10% glycerol v/v. The crystals belong to space group P2₁2₁2₁, have unit cell dimensions a=46, b=57, c=66 Å, and display significant non-isomorphism. between specimens, despite reproducible unit cell dimensions. Therefore native and isomorphous derivative data were collected from single crystals that were subjected to sequential rounds of cryocooling, data collection thawing, and metal soaks. Two derivatives were prepared for this protein mutant: the first by soaking a crystal in cryobuffer containing 2 mM of the mercurial reagent PSMB after native data was collected, and the second by soaking in a 1 mM K₂PtCl₄. The native data and mercury derivative were collected to 2.2 Å resolution on an in-house RAXIS-IV area detector, while the platinum derivative data were collected to 1.7 Å resolution at the ALS on beamline 5.0.2, using an incident wavelength of 1.07 Å, corresponding to the platinum anomalous edge. An additional S2296C native data set was collected to 1.5 Å resolution at Brookhaven NSLS beamline X-26C and used for the final refinement. All data were processed using DENZO/SCALEPACK and merged using the CCP4 program suite, and phases were calculated and refined using SHARP, SOLOMON, and 2-D histrogram matching. Model building was performed using O, and the structure was refined using XPLOR 3.8 after removing 5% of the measurements in order to monitor the free R-factor. The final R_{cryst} is 20.1%, and the R_{free} is 22.5%. The final refined model of the protein domain consists of 158 amino acid residues and 194 water molecules. The stereochemical quality of the protein model was examined throughout the refinement using PROCHECK The final model contains no residues with disallowed backbone dihedral angles. Data and refinement statistics are shown in Table. 1.

TABLE 1
Data and Refinement Statistics

| | | Native 1 | Hg | Pt | Native 2 |
|---|---|---|---|---|---|
| Diffraction Data | | | | | |
| Resolution (Å) | | 2.2 | 2.2 | 1.7 | 1.5 |
| Source | | RAXIS-IV | RAXIS-IV | ALS 5.0.2 | NSLS X-26C |
| Space Group | | P2₁2₁2₁ | P2₁2₁2₁ | P2₁2₁2₁ | P2₁2₁2₁ |
| Unit Cell: | a | 45.9 | 45.8 | 46.2 | 46.4 |
| | b | 57.0 | 57.0 | 57.2 | 56.4 |
| | c | 66.2 | 66.3 | 66.2 | 65.7 |

| | | | | |
|---|---|---|---|---|
| Wavelength (Å) | 1.54 | 1.54 | 1.07 | 1.01 |
| No. refl.(unique) | 7938 | 8134 | 19711 | 27232 |
| Redundancy | 5.3 | 4.8 | 6.1 | 4.5 |
| Completeness¹ | 97.0 (94.6) | 99.6 (97.3) | 91.2 (83.4) | 96.0 (94.0) |
| R_{merge} (%) | 4.2 | 5.6 | 3.6 | 4.0 |
| MIR Phasing | | | | |
| Number of sites | | 1 | 1 | |
| Rᵢₛₒ, Kₑₘₚ | | 18.2, 6.6 | 18.0, 11.5 | |
| Phasing power² | | 1.2/1.3/0.9 | 0.9/1.0/1.9 | |
| R_{Cullis} | | 0.8/0.8/0.9 | 0.8/0.9/0.6 | |

| Overall FOM³ | | 0.36/0.31 | |
|---|---|---|---|
| Refinement | | | |
| Resolution Range | 50.0-2.2 | | 10.0-1.5 |
| R_{cryst} | 20.4 | | 20.1 |
| R_{free} | 26.0 | | 22.5 |
| Protein Atoms | 1135 | | 1035 |
| Solvent Atoms | 104 | | 194 |
| Ramachandran | | | |
| Distribution | 0.005, 1.389 | | |
| (% core, allowed, | | | |
| generous, dissallowed) | | | |
| rms bonds, angles | | | |
| Average Protein | | | |
| B-factors | | | |

¹Completeness reported for all reflections and for highest resolution 0.1 Å resolution shell.
²Phasing Power and R_{Cullis} reported for centric isomorphous differences, acentric isomorphous differences, and acentric anomalous differences, respectively.
³Overall FOM reported for acentric and centric reflections, respectively.

Crystals of the fully wild-type C2 were grown from 20% PEG 8000, 0.1 M CAPS (pH 10.5) 0.15 M NaCl, protein 14 mg/ml. These crystals belong to space group P2₁2₁2₁ and have unit cell dimensions a=49 Å, b=57 Å, c=77 Å. These data were used to determine the structure of the wild-type C2 domain by molecular replacement, using the S2296C structure as a model. The structure was refined as described above. The structures of wild type and S2286C protein are virtually identical. Finally, data were collected on an S2296C protein/phospholipid complex after soaking crystals with 10 mM glycerophosphoserine, which is an analogue of the nature phosphatidylserine lipid that is bound by factor VIII. The structure of the complex was determined by difference Fourier analysis and subsequently refined as described above.

Results

The secondary structure and overall fold of the C2 domain is shown in FIGS. 2 and 3. The domain contains 12 β-strands, eight of which form a core β-sandwich structure. The overall dimensions of this core structure are approximately 35 Å long by 25 to 30 Å wide, with the longer axis parallel with the axis of the barrel formed by the β-sheets. The structure and orientation of the β-strands found in the protein core is quite similar to the structure predicted based on sequence threading and homology modeling against galactose oxidase lipid binding domain. The structure of the C2 domain backbone is elongated by approximately 10 Å beyond this core fold by the extension of two β-strands (6 and 7) beyond the sandwich structure, and by the presence of two additional anti-parallel β-strands (3 and 4) at the same end of the protein fold. These elements of structure were not predicted by homology modeling, and in general the structure of the protein outside the β-sandwich core is significantly different from that study.

In addition to the βsheets that comprise the majority of the protein structure, there are two short regions of 3 ₁₀ helix near the N-terminus of the protein, but no standard α-helices. The N- and C-terminal regions of the domain are linked by a disulfide bridge between residues 2174 and 2326, and there is one observable cis-peptide bond, corresponding to Pro 2299.

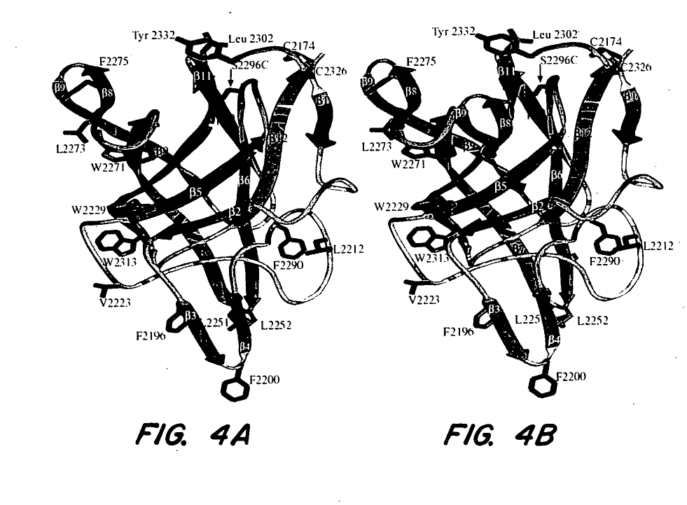

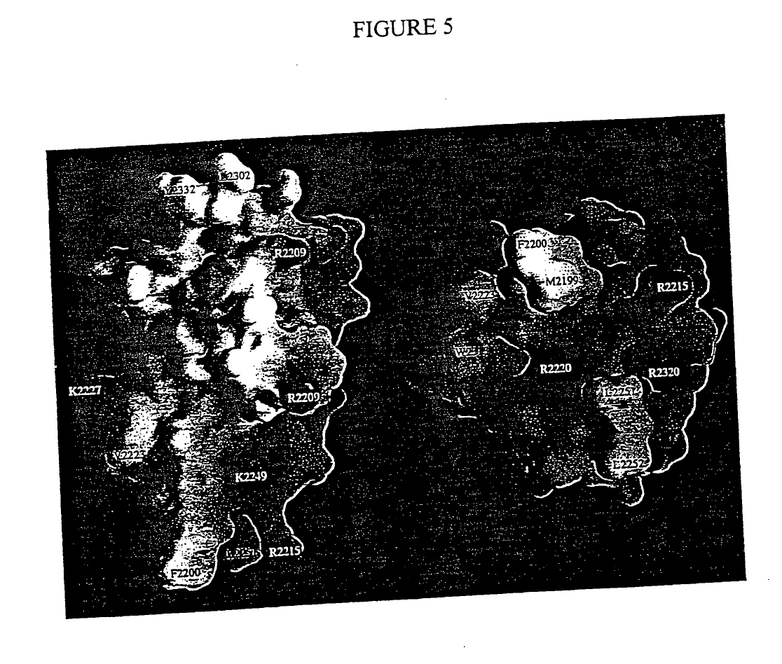

The protein structure exhibits two significant regions of exposed hydrophobic surface. The first, at the upper end of the β-sandwich, includes Phe 2275, Tyr 2332 and Leu 2302. The second surface is formed by two β-turns and a loop as shown in FIGS. 4 and 5. The residues connecting strands β3 and β4 form a β-turn, and present Met 2199 and Phe 2200 to the solvent. The total accessible surface area of these two residue is approximately 335 Å². In contrast, the residues connecting strands 6 and 7 form a type II β-turn and place Leu 2251 and 2252 within the same solvent-exposed surface; each side chain contributes approximately 160 Å² of accessible surface area to the protein structure in this region. In addition, Val 2223 extends from the loop that directly precedes strand 5 into this non-polar surface and is also found to be highly solvent accessible (84 Å²). As shown in FIGS. 4 and 5, these side-chains extend beyond the protein core and form a collection of non-polar residues that appear appropriate for burial in the lipid bilayer. Surrounding this hydrophobic region is a ring of at least four basic residues (Arg 2215, Arg 2220, Lys 2227 and Lys 2249) that could further promote association with exposed phospholipid bilayers by interacting with anionic lipid headgroups such as phosphatidylserine. Based on the crystal structure, it is estimated that the free energy of membrane association is the result of at least five favorable transfer energies of non-polar amino acid side-chains to a non aqueous environment (two leucines, one valine, one methionine and one phenylalanine) and four additional electrostatic interactions between basic side chains and anionic phospholipid head groups. Depending on the precise orientation of the protein domain in the lipid bilayer and the depth of penetration of individual side chains, such favorable interactions can provide in excess of 10 to 20 kcal/mol binding energy.

A previous study has reported that a 21-residue peptide from the C2 domain, corresponding to residues 2303 to 2323 near the carboxy-terminus, competes with factor VIII for membrane-binding in vitro. It was shown that this peptide assumes an amphipathic helical structure in the presence of detergent micelles, leading to the hypothesis that a similar structure might be formed by these residues within the protein domain and thereby participate in membrane binding. In the structure of the C2 domain, these residues are observed to particpate in the structure of the β-sandwich core and correspond to β-strands 11 and 12, and it is unlikely that refolding of this region to form an alpha helix would be induced by membrane binding.

There are currently seventeen residues in the factor VIII C2 domain that have been reported as sites of deleterious individual point mutations in patients with hemophilia A. It is possible to catalogue these mutations into several groups on the basis of their observed biological effects and positions in the protein structure. Of these residues, it is interesting to note that only one (Val 2223) is located in the region predicted to directly participate in membrane binding. This might indicate that the protein displays a reduced, but still effective binding affinity to exposed phospholipid bilayers when Val 2223 or other individual side chains in this interface are eliminated, so that point mutations in this region result in relatively asymptomatic individuals. In contrast, mutations in the protein core or at the surfaces that interact with the C1 domain or with von Willebrands factor might interfere with protein production or increase the rates of degradation and of clearance from the serum, resulting in more profound physiological effects. Of the reported C2 point mutations in hemophiliacs, eight (Ile 2185, Ile 2190, Ala 2192, Thr 2245, Phe 2260, Ile 2262, Gly 2285 and Gly 2325) appear to be directly involved in packing the protein core. Of these all except Ile 2185 and Gly 2285 exhibit reduced protein levels and moderate to severe bleeding defects. Two additional side chains, Arg 2209 and Arg 2246, are involved in structural hydrogen-bonding networks that also appear to stabilize the protein fold. Two residues (Met 2238 and Pro 2300) appear to be located at the surface. Perhaps most interestingly, three exposed residues (Trp 2229 from strand 5 and Arg 2304 and 2307 from strand 11) are clustered at a common surface distal to both the putative membrane association region and the other hydrophobic interface and do not appear to be critical for protein folding. Mutations of all three of these residues are associated with mild to moderate effects on coagulation. It is possible that this surface represents the binding site for another coagulation protein, such as von Willebrands factor (vWF). A mutation that interferes with the association between factor VIII and vWF would display a similar phenotype to a directly destabilizing mutation, as free factor VIII is proteolytically cleared from the serum in the absence of bound vWF.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

What is claimed is:

1. A crystal of a protein-ligand complex comprising a protein-ligand complex of an N-terminal truncated factor VIII and a ligand, wherein the crystal effectively diffracts X-rays for the determination of the atomic coordinates of the protein-ligand complex to a resolution of greater than 5.0 Angstroms; and wherein the N-terminal truncated factor VIII:
(a) lacks at least 2000 amino acids from the flexible N-terminus of the corresponding full-length factor VIII; and
(b) retains the C2 domain of the corresponding full-length factor VIII.

2. The crystal of claim 1, wherein the ligand comprises a phospholipid.

3. The crystal of claim 1, wherein the N-terminal truncated factor VIII comprises an amino acid sequence of amino acids 2174 to 2326 of SEQ ID NO:1, or an amino acid sequence that differs from amino acids 2174 to 2326 of SEQ ID NO:1 by only having conservative substitutions.

4. The crystal of claim 1, wherein the ligand is glycerophosphorylserine.

5. The crystal of claim 1, having space group of P2₁2₁2₁ and a unit cell of dimensions of a=46, b=57, and c=66 Angstroms.

6. The crystal of claim 1, wherein the N-terminal truncated factor VIII has secondary structural elements that include an eight-stranded, antiparallel β-barrel arranged in the order: β-sheet (1), β-sheet (2), β-sheet (3), β-sheet (4), β-sheet (5), β-sheet (6), β-sheet (7), β-sheet (8).

7. An N-terminal truncated factor VIII lacking from 2000 to 2200 of the first N-terminal amino acids of the corresponding full-length factor VIII.

8. The N-terminal truncated factor VIII of claim 7, comprising an amino acid sequence of amino acids 2174 to 2326 of SEQ ID NO:1, or an amino acid sequence that differs from amino acids 2174 to 2326 of SEQ ID NO:1 by only having conservative substitutions.

9. The N-terminal truncated factor VIII of claim 7, having a selenomethionine that has been substituted for a methionine in said amino acid sequence.

10. The N-terminal truncated factor VIII of claim 7, having an amino acid sequence of amino acids 2169 to 2332 of SEQ ID NO:1, or an amino acid sequence that differs from amino acids 2169 to 2332 of SEQ ID NO:1 by only having conservative substitutions.

11. A method of using the crystal of claim 1 in a drug screening assay, comprising:
(a) selecting a potential ligand by performing structure-based drug design with the three-dimensional structure determined for the crystal, wherein said selecting is performed in conjunction with computer modeling;
(b) contacting the potential ligand with the ligand binding domain of factor VIII; and
(c) detecting the binding of the potential ligand for the ligand binding domain; wherein a potential drug is selected on the basis of its having a greater affinity for the ligand binding domain of factor VIII than that of a standard ligand for the ligand binding domain of factor VIII.

12. The method of claim 11, wherein the standard ligand is glycerophosphorylserine, phosphate or sulfate.

13. A method of using N-terminal truncated factor VIII to grow a crystal of a protein-ligand complex, comprising:
(a) contacting the N-terminal truncated factor VIII with a ligand, wherein the N-terminal truncated factor VIII forms a protein-ligand complex with the ligand; and
(b) growing the crystal of the protein-ligand complex; wherein the crystal effectively diffracts X-rays for the determination of the atomic coordinates of the protein-ligand complex to a resolution of greater than 5.0 Angstroms.

14. The method of claim 13, wherein said growing is performed by sitting-drop vapor diffusion.

15. The method of claim 13, wherein said ligand is glycerophosphorylserine.
